(19) United States
(12) Patent Application Publication
(10) Pub. No.: US 2005/0281718 A1
(43) Pub. Date: Dec. 22, 2005

(54) Electric air freshener for a vehicle

(76) Inventor: Elizabeth Echevarria, Tampa, FL (US)

Correspondence Address: Elizabeth Echevarria, P.O. Box 22202, Tampa, FL, 33622, US

(21) Appl. No.: 11/115,674

(22) Filed: Apr. 27, 2005

Related U.S. Application Data
(60) Provisional application No. 60/566,687, filed on Apr. 30, 2004.

(51) Int. Cl. A61L 9/03

(52) U.S. Cl. 422/125

(57) ABSTRACT

An electronic air freshener for a vehicle includes a housing having a plug extending from the rear surface thereof for insertion into a vehicle cigarette lighter. The housing includes an interior chamber that receives a scented insert. The insert rests on a heating element that is powered by the cigarette lighter. Dispensing apertures are positioned on the front surface of the housing. The housing further includes a receptacle to which an auxiliary electronic device may be coupled allowing it to be used simultaneously with the air freshener. When the device is inserted into the cigarette lighter, the heating element vaporizes the scented insert and disperses it through the apertures to deodorize a vehicle passenger compartment.

### BACKGROUND OF THE INVENTION

The present invention relates to an air freshener that is activated by a vehicle cigarette lighter to automatically disperse a scented fragrance within a vehicle passenger compartment.

### DESCRIPTION OF THE PRIOR ART

Vehicle passenger compartments are notorious for harboring undesirable odors. Numerous vehicle air fresheners exist in the prior art, most of which employ an aerosol spray canister for dispensing an air freshener to the passenger compartment. However, the effective duration of such air fresheners is limited. The present invention provides a uniquely configured air freshener for a vehicle that is powered by a vehicle cigarette lighter for gradually and continuously dispersing a deodorizing agent. Furthermore, the device includes an electrical receptacle thereon allowing an additional electronic device to be used during operation of the air freshener.

### SUMMARY OF THE INVENTION

The present invention relates to an electric air freshener for a vehicle. The device comprises a housing having an interior chamber. Extending from a rear surface of the housing is a male electrical plug for insertion into a vehicle cigarette lighter. Received within the interior chamber is an elongated, aluminum heating element that is powered by the vehicle cigarette lighter when the plug member is properly coupled therewith. A scented insert is removably received within the interior chamber and rests on the heating element; when the heating element is activated, the insert is vaporized and dispensed through apertures on the housing to deodorize the vehicle passenger compartment. Also positioned on the front surface of the housing is a receptacle for receiving a plug from an auxiliary electrical device such as a cell phone, radio, or other device allowing it to be operated simultaneously with the air freshener.

It is therefore and object of the present invention to provide an air freshener for a vehicle that gradually and effectively emits a deodorizing agent to the passenger compartment.

It is another object of the present invention to provide of vehicle air freshener that allows a user to couple an auxiliary electronic device therewith.

Other object, features, and advantages of the present invention will become readily apparent from the following detailed description of the preferred embodiment when considered with the attached drawings and the appended claims.

### BRIEF DESCRIPTION OF THE DROWINGS

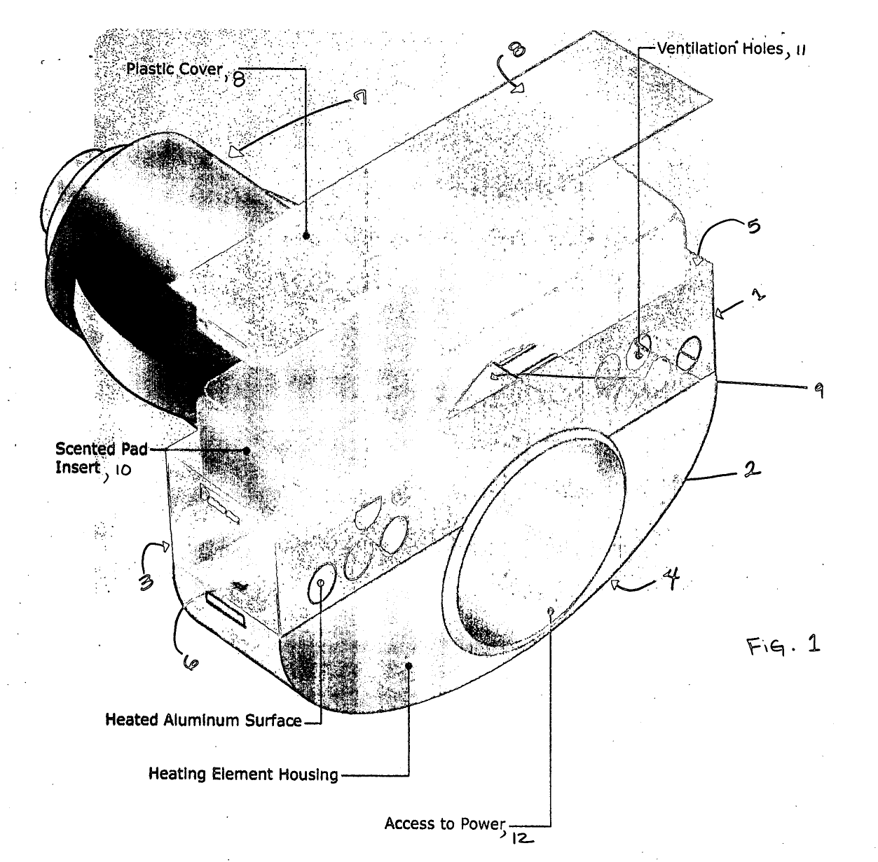

FIG. 1 is a perspective view of the electric air freshener according to the present invention.

FIG. 2 is a perspective view of the air freshener coupled with the vehicle cigarette lighter.

### DESCRIPTION OF THE PREFERRED EMBODIMENT

The present invention relates to an electronic air freshener for a vehicle. The device comprises a housing 1 including a front surface 2, a rear surface 3, an arcuate, contoured lower surface 4 and an open top 5 in communication with an interior chamber 6. Extending from the rear surface of the housing is a male electrical plug 7 for insertion into a vehicle cigarette lighter.

An elongated removable cover 8 provides selective access to the interior chamber. Received within the interior chamber is an elongated, aluminum heating element 9 that is powered by the vehicle cigarette lighter when the plug member is properly coupled therewith. A scented insert 10 is removably received within the interior chamber and rest on the heating element; when the heating element is activated, the insert is vaporized and dispensed through apertures 11 on the front surface of the housing to deodorize the vehicle passenger compartment. Also positioned on the front surface of the housing is a receptacle 12 for receiving a plug member from an auxiliary electrical device such as a cell phone, radio, or other device allowing it to be operated simultaneously with the air freshener.

The above described device is not limited to the exact details of construction and enumeration of part provided herein. Furthermore, the size, shape and materials of construction can be varied.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

1. An electric air freshener for a vehicle comprising:
a housing having a lower surface and an openable top in communication with an interior chamber, and exterior apertures;
said housing having a male electrical plug for insertion into a vehicle cigarette lighter;
said interior chamber having a heating element in electrical communication with said male electrical plug;
and said interior chamber having a scented insert juxtaposed to said heating element;
so that upon plugging in said male electrical plug into an electrified vehicle cigarette lighter, said heating element is activated, heating said scented inset, which upon vaporization scents a vehicle having said vehicle cigarette lighter.

2. The electrical air freshener according to claim 1, further comprising:
said housing having a receptacle for receiving a plug from an auxiliary electrical device to enable simultaneous operation of said auxiliary electrical device and said air freshener.

3. An electric air freshener for a vehicle comprising:
a housing containing interiorly a means for containment of a means for scenting;
a means for heating in said housing having electrical communication with a male electrical plug for insertion into a vehicle cigarette lighter;
said housing further having apertures enabling scents from said means of scenting to escape upon plugging in said plug into an electrically activated vehicle cigarette lighter and the consequent heating by said heating element of said means of scenting.

4. The electrical air freshener according to claim 4, further comprising:
said housing having a receptacle for receiving a plug from an auxiliary electrical device to enable simultaneous operation of said auxiliary electrical device and said air freshener.
